(19) United States
(12) Patent Application Publication
(10) Pub. No.: US 2004/0151754 A1
(43) Pub. Date: Aug. 5, 2004

(54) Steroid suspensions for intraocular use

(75) Inventor: Paul Ashton, Boston, MA (US)

Correspondence Address: ROPES & GRAY LLP, ONE INTERNATIONAL PLACE, BOSTON, MA, 02110-2624, US

(73) Assignee: Control Delivery Systems, Inc., Watertown, MA (US)

(21) Appl. No.: 10/742,042

(22) Filed: Dec. 19, 2003

Related U.S. Application Data
(60) Provisional application No. 60/435,088, filed on Dec. 20, 2002.

(51) Int. Cl. A61K 9/00

(52) U.S. Cl. 424/427

(57) ABSTRACT

The subject invention relates to methods and compositions of steroid suspensions suitable for intraocular use in the treatment or prevention of a variety of ocular diseases. Specifically, the invention provides pharmaceutical compositions with significantly reduced endotoxin levels that are suitable for intraocular use. The invention also relates to methods of reducing the level of endotoxins within certain compositions, such as pharmaceutical compositions, that can be used for intraocular delivery.

### RELATED APPLICAITONS

This application claims the benefit of U.S. patent application Ser. No. 60/435088, filed Dec. 20, 2002, the specification of which is incorporated by reference herein.

### BACKGROUND OF THE INVENTION

One of the most important problems in clinical ophthalmology is the unwanted proliferation of intraocular and epibulbar tissue such as retinal pigment epithelium cells in the case of proliferative vitreoretinopathy; vascular cells in eyes with ischaemic retinopathies such as proliferative diabetic retinopathy or proliferative retinopathy occurring after central retinal vein occlusion; lens epithelium cells in eyes developing secondary cataract; and fibroblastic cells in patients after antiglaucomatous filtering surgery. Proliferation of intraocular and episcleral cells is often accompanied and stimulated by intraocular inflammation. Corticosteroids are known to reduce intraocular inflammation and, depending on their concentration, to suppress proliferation of cells. Consequently, they have been used in many ocular conditions given either locally or systemically. To achieve the highest concentration of a drug at its site of action, however, it is best to give it directly into the region of required action. This ensures highest possible concentrations and fewer side effects for the rest of the body. For instance, the corticosteroid can be delivered intraocularly, e.g., by injection or implantation in the form of a sustained release device.

Triamcinolone acetonide has been used locally as a periocular injection for the treatment of cystoid macular edema secondary to uveitis or as a result of intraocular surgery. Intravitreal corticosteroids have also been tried experimentally in the prevention or treatment of proliferative vitreoretinopathy, retinal neovascularization, choroidal neovascularization, vein occlusion, diabetic retinopathy, diabetic macular edema, retinal pigmentosa, uveitis, and edema. The safety of intravitreal corticosteroids has been supported by prior animal studies and human trials. However, in a certain number of instances, complications to this procedure include endophthalmitis. In the literature, it has been speculated that infectious endophthalmitis may be the result of the locally immunosuppressive effect of corticosteroid treatment.

### SUMMARY OF THE INVENTION

The present invention relates to the discovery that endotoxin levels in commercial preparations of steroids, while within FDA limits for endotoxin contamination, can result in intraocular levels of endotoxins that are sufficient to produce hypopyon or pseudo endophthalmitis conditions.

One aspect of the invention provides a packaged pharmaceutical comprising: (1) a pharmaceutical composition formulated for intraocular injection or implantation as a sustained release device, which composition includes an amount of a steroid sufficient for use in treating or preventing an ocular disorder, and which pharmaceutical composition has an endotoxin concentration of less than 0.3 EU/mL and preferably less than 0.03 EU/mL, and even more preferably less than 0.01 EU/mL; and (2) a label and/or instructions for use of the pharmaceutical composition or device in the treatment or prevention of said ocular disorder.

Another aspect of the invention provides a use of a low endotoxin steroid composition in the manufacture of a medicament for the treatment or prevention of an ocular disorder, which steroid composition has an endotoxin concentration of less than 0.3 EU/mL and preferably less than 0.03 EU/mL, and even more preferably less than 0.01 EU/mL; and is formulated for intraocular injection or implantation as a sustained release device.

Yet another aspect of the invention provides a method for treating or preventing an ocular disorder comprising administering to a patient's eye by intraocular injection or implantation of a sustained release device, a steroid composition having an endotoxin concentration of less than 0.3 EU/mL, and preferably less than 0.03 EU/mL, and even more preferably less than 0.01 EU/mL.

The ocular disorders that can be treated or prevented by the instant invention include, but are not limited to: cancerous primary tumors in the eye (e.g., retinoblastoma); ocular neovascularization; ocular edema; ocular inflammation; chronic pain in the eye; endogenous uveitis; Behcet's Disease; corneal transplantation; vernal keratoconjunctivitis; ligneous keratoconjunctivitis; dry eye syndrome; anterior uveitis; onchocerciasis; diseases of the retina; diseases of the retinal pigment epithelium and choroid; retinal degeneration; diabetic retinopathy; closed angle (acute) glaucoma; open angle (chronic) glaucoma; congenital glaucoma; secondary glaucoma; retinal detachment; sickle cell retinopathy; senile macular degeneration; retinal neovascularization; subretinal neovascularization; rubeosis iritis; inflammatory diseases; chronic posterior and panuveitis; neoplasms; pseudoglioma; neovascular glaucoma; neovascularization resulting or following a combined vitrectomy and lensectomy; vascular diseases; retinal ischemia; choroidal vascular insufficiency; choroidal thrombosis; neovascularization of the optic nerve; diabetic macular edema; cystoid macular edema; macular edema; retinitis pigmentosa; retinal vein occlusion; proliferative vitreoretinopathy; angioid streak; retinal artery occlusion; and neovascularization due to penetration of the eye or ocular injury.

In certain embodiments, the steroid is a corticosteroid, such as dexamethasone, prednisolone, fluocinolone or fluocinolone acetonide, triamcinolone or triamcinolone acetonide, loteprednol etabonate, cortisone, or flumetholone, or analogs, derivatives, pharmaceutically acceptable salts, esters, prodrugs, codrugs, or protected forms thereof. In preferred embodiments, the steroid is triamcinolone acetonide, fluocinolone acetonide, or loteprednol etabonate.

In one embodiment, the composition is a solution or flowable liquid (polymer or hydrogel) for intraocular injection. In other embodiments, the composition is an implantable sustained release device, dimensioned for implantation in the eye.

In certain embodiments, the subject steroid compositions are formulated to deliver an effective dose of steroid in 500 μL, preferably 100 μL, 50 μL, or less.

In certain embodiments, the subject steroid compositions have an endotoxin contamination which is less than 0.1 EU/mg steroid, preferably less than 0.01 EU/mg steroid, or even more preferably less than 0.005 EU/mg steroid.

In certain embodiments, the composition or sustained release device further comprises one or more endotoxin inhibitors.

In one embodiment, the composition is co-formulated or conjointly administered with one or more endotoxin inhibitors.

### DETAILED DESCRIPTION OF THE INVENTION

(i) Overview

The use of intravitreal injections of triamcinolone and other steroids has become widespread in recent years. Despite this general practice, however, there are disadvantages to administering steroids to the eye. One of the problems appears to be the development of hypopyon or a pseudo-endophthalmitis shortly after administration. The present invention relates to the discovery that endotoxin levels in commercial preparations of steroids, while within FDA limits for endotoxin contamination, result in intraocular levels of endotoxins that are significantly higher and produce these unwanted effects.

To illustrate, a 72 year old male patient underwent injection of 0.1 mL (4 mg) of Kenalog (triamcinolone acetonide) for chronic cystoid macular edema with vision of count fingers. Forty-eight hours after injection, the patient's vision had decreased to hand motion and he was found to have marked anterior chamber and vitreous cell inflammation with a small hypopyon, prominent vitreous haze, and debri. He thereafter underwent a vitreous tap with an injection of vancomycin and Fortaz®. Fourteen days later, the patient's vitreous and anterior chamber inflammation had completely cleared. The endotoxin level of the Kenalog administered to this patient was assayed by the gel-clot method (as a limits test with comparison to the maximum allowed FDA limit, as set forth in the Guideline for Validation of Limulus Amebocyte Lysate Test as an End-Product Test for Human and Animal Parenteral Drugs, Biological Products, and Medical Devices). Results showed that the endotoxin concentration in the Kenalog was less than 0.03 EU/mL at a 1:100 dilution of the suspension, equaling a level of less than 3.0 EU/mL in the undiluted product used in the study.

The presence of endotoxins in biologically derived products (biologicals) prepared for therapeutic use is of major concern due to the diverse and potentially harmful biological activities of these molecules. Maintaining sterility in processes used in the manufacture of biologicals, together with stringent protocols for the preparation of equipment, helps to ensure that products have acceptably low levels of endotoxins. The FDA's specification for maximum endotoxin level of triamcinolone acetonide is 4.39 EU/mL. The Kenalog assayed in the study described above was well below this level. The FDA's specification, however, is based on 5 EU as the maximum safe endotoxin exposure for a 70 kg individual. This suggests that a safe systemic exposure is approximately 0.001 EU/mL. For an intraocular injection, however, where it can be expected that the endotoxin will not be distributed throughout the body, 0.1 mL of Kenalog (as tested here) would result in an intravitreal endotoxin concentration of approximately 0.1 EU/mL. Such a concentration may cause endotoxinmediated side effects. The above-described patient may have had endophthalmitis, which may have resulted from the intraocular injection of Kenalog. Presently, there are no FDA guidelines concerning the maximum endotoxin level for an intraocular dose of triamcinolone. It is possible that, while 4.39 EU/mL is an appropriate maximum concentration for general use, it is too high a level for an intraocular injection.

The vitreous acts as a superb medium for bacteria growth. In fact, in the past, animal vitreous was used as a culture medium. Bacteria, as foreign objects, incite an inflammatory response. The cascade of inflammatory products increases breakdown of the blood-ocular barrier, as well as inflammatory cell recruitment. Damage to the eye occurs from the breakdown of the inflammatory cells releasing the digestive enzymes and the possible toxins produced by the bacteria. Destruction occurs at all tissue levels that are in contact with the inflammatory cells and toxins.

In recognizing this problem, the present invention provides formulations of intraocular steroids which have been purified such that, when injected into the eye, they produce a vitreal endotoxin concentration of less than 0.01 EU/mL, and even more preferably less than 0.001 EU/mL. In certain embodiments, the injected steroid compositions are provided in the form of a solution or suspension which is intended for injection in volumes of less than 500 μL, and even more preferably less than 200 μL, less than 100 μL, or even less than 50 μL. Accordingly, the subject intraocular steroid formulations include solutions and suspensions having endotoxin concentrations of 0.3 EU/mL or less, and even more preferably of 0.03 EU/mL or less. That is, the subject formulations have endotoxin concentrations significantly less than FDA levels permitted for triamcinolone preparations.

In certain embodiments, the subject steroid compositions have an endotoxin contamination which is less than 0.1 EU/mg steroid, and even more preferably less than 0.01 EU/mg steroid, or less than 0.005 EU/mg steroid.

The present invention also provides packaged pharmaceuticals including a low endotoxin content formulation of a steroid suitable for intraocular injection, e.g., in volumes of less than 500 μL or less, and a label and/or instructions for use in treating or preventing an ocular disorder.

Other aspects of the invention provide formulations of intraocular steroids which have been co-formulated with one or more agents that inhibit the effect of endotoxins, such as cyclooxygenase inhibitors, cyclosporins, etc.

(ii) Definitions

The term “endotoxin” is used herein to refer to glycolipids and their metabolites found in the outer membrane of gram-negative bacteria. Endotoxins can be assayed, for example, using a Limulus Amoebocyte Lysate (LAL) test kit (Pyrogent Plus, Bio-Whittaker, Cat. No. N284).

The term “patient,” as used herein, refers to either a human or a non-human animal.

The term “preventing” is art-recognized, and when used in relation to a condition, such as a local recurrence (e.g., pain), a hyperproliferative disorder, or any other medical condition, is well understood in the art, and includes administration of a composition that reduces the frequency of, or delays the onset of, symptoms of a medical condition in a patient relative to a patient who does not receive the composition. Thus, for example, prevention of retinopathy includes reducing the number of detectable retinopathies in a population of patients receiving a prophylactic treatment relative to an untreated control population, and/or delaying the appearance of detectable retinopathies in a treated population versus an untreated control population, e.g., by a statistically and/or clinically significant amount. Prevention of an infection includes, for example, reducing the number of diagnoses of the infection in a treated population versus an untreated control population, and/or delaying the onset of symptoms of the infection in a treated population versus an untreated control population. Prevention of pain includes, for example, reducing the frequency of, or alternatively delaying, pain sensations experienced by patients in a treated population versus an untreated control population.

By “sustained release device” it is meant a device that releases drug over an extended period of time in a controlled fashion. Examples of sustained release devices useful in the present invention may be found in, for example, U.S. Pat. Nos. 5,378,475, 5,773,019, 6,001,386, 6,217,895, 6,548,078, 6,375,972, 5,902,598, and 6,331,313, and U.S. patent application Ser. No. 10/714,549, the entire contents of which are incorporated by reference herein.

The term “treatment,” means reversal, alleviation, amelioration, reduction, inhibition, prevention, stabilization, prophylaxis, relief of, or cure of a disease, disorder, or condition. Exemplary, non-limiting disease symptoms include pain and inflammation. Exemplary, non-limiting disease conditions include osteoarthritis, rheumatoid arthritis, neoplasia, microbial infection, and angiogenesis.

The phrase “therapeutically effective amount” as used herein means that amount of a compound, material, or composition comprising a compound of the present invention which is effective for producing some desired therapeutic effect at a reasonable benefit/risk ratio applicable to any medical treatment.

By “vitreous” of the eye, it is meant the vitreous or vitreal cavity of the eye. By “aqueous” of the eye, it is meant the aqueous humor of the eye.

(iii) Methods of Removal of Endotoxins

There are a number of methods that can be used to remove endotoxins from the subject pharmaceutical preparations. Suitable methods of minimizing endotoxin contamination during purifications include (i) using sterile solutions and equipment to minimize the introduction of microorganisms, (ii) using filtration during purification, often with 0.45 micron or 0.22 micron filters, to remove micro-organisms, (iii) working at low temperature to minimize microbial growth, and (iv) adding bacteriostatic agents to the purification. Obviously the practicality of these methods depends on the specifics of the purification. Except for sterile purification techniques (i.e., ones with all sterile components and performed in a sterile environment), some microbes are often present. As a result, endotoxins can be present in the final product. Since the cost of sterile purification is often prohibitive, it may be desirable to have a method of separating the pure product from endotoxins as the final step. Some of these methods for removing endotoxins are discussed below.

The ease of separating endotoxins from the steroid product can depend in part on the similarities or differences between the endotoxins and the steroid. For example, positively charged filters of various kinds have been widely used to remove negatively charged endotoxins, but such a separation cannot be utilized with a negatively charged steroid having characteristics similar to the endotoxins. Additionally, endotoxins have a low density of negative charge, which can make removal inefficient.

Size based fractionation techniques, like gel permeation, chromatography, or ultrafiltration, can also be utilized to separate endotoxins and steroid product.

Affinity chromatographic methods can also be used, where endotoxins are removed by adsorption to the peptide antibiotic polymyxin B.

U.S. Pat. No. 6,365,147 (the entire content of which is incorporated herein by reference) describes methods for removing endotoxins from biological solutions using immobilized metal affinity chromatography. Such methods can be used to prepare low endotoxin steroid preparations. Specifically, the patent relates to methods for depleting endotoxins from biological solutions by exposure of solutions containing endotoxins to an immobilized metal affinity chromatography matrix composed of a metal ion such as iron (III) bounds to a resin, wherein the metal is capable of selectively binding endotoxins in the solution.

Wei et al. (Chromatography 23(2): 79-84, 2002, fully incorporated by reference) describe the use of three types of affinity membranes for endotoxin removal from a number of biological samples, including from medical injections, such as injections containing steroids. The three types of membranes used are chitosan affinity membrane (KFCC517), hydrophobic and cationic charged membrane (KFCG316), and metal chelate affinity membrane (KFCM402). Under optimal conditions, the removal efficiency of endotoxins from the hydrocortisone medical injection is reportedly greater than 94% (from 1 EU/mL before filtration to 0.059 EU/mL after filtration), while the effective component hydrocortisone was recovered at 100%. It is expected that repeated use of the same procedure may reduce the endotoxin level further without much loss of the effective component given its high retention rate (100%). This also illustrates that the separation of endotoxins from steroids, which do not generally contain many cationic charges on their structure, will be generally quite easy to achieve using these membranes. It is also possible to modify the separation conditions, such as the pH, ionic strength, and flow rate used to achieve optimal recovery of the effective component while remaining maximal removal of endotoxins. Such conditions are discussed by Wei et al.

Commercially available kits, such as the Detoxi-Gel Endotoxin Removal Gel from Pierce Biotechnology, can also be used to remove endotoxions from steroid solutions. It is reported that endotoxin removal efficiency of the gel is greater than 99% in one pass.

Selective binding of endotoxins on charged, hydrophobic or affinity media, or separation on the basis of size can also be performed. At pH levels greater than pH 2, endotoxin aggregates are negatively charged and will bind to positively charged surfaces such as asbestos or anion exchangers. Endotoxins will also bind to aliphatic polymers such as polypropylene, polyethylene, polyvinylidene fluoride, polytetrafluoroethylene, and hydrophobic chromatographic systems via hydrophobic interactions. Endotoxins can also be specifically removed by affinity chromatography using immobilized polymicin B. Additionally, because endotoxins exist primarily as large molecular weight complexes, they can often be removed from desired components by ultrafiltration or gel filtration methods.

Other approaches which can be used to destroy or remove endotoxins include hydrolysis with acid or base, oxidation, alkylation, and/or heat treatment.

The methods described above are for illustrative purpose only, and are by no means limiting. Many other art-recognized methods can be used to remove endotoxins from biological samples, such as the subject steroid solutions or suspensions for pharmaceutical use. It is also contemplated that removal of endotoxins from biological samples, such as the subject steroid composition, can be achieved using a combination of more than one of the methods described above, and each method can be repeatedly used independent of the other methods.

(iii) Conjoint Administration and Co-Formulation of Endotoxin Inhibitors

Instead of, or in addition to, purification of the intraocular steroid formulation, the present invention also contemplates the use of other agents which can inhibit the effect of endotoxins (endotoxin inhibitors or antagonists), e.g., which can be co-formulated or otherwise conjointly administered with the intraocular steroid compositions of the present invention.

In certain embodiments, the subject intraocular steroids are combined with a cyclooxygenase (COX) inhibitor, especially a COX-2 inhibitor. Prostaglandins play a major role in the endotoxin-induced inflammation process. In inflammatory conditions, COX-2 is rapidly induced by cytokines, growth factors, and bacterial endotoxins. NSAIDs have been found to prevent the production of prostaglandins by inhibiting enzymes in the human arachidonic acid/prostaglandin pathway, including COX.

In certain preferred embodiments, the subject intraocular steroids are co-formulated or at least co-delivered with a selective COX-2 inhibitor. The phrase “cyclooxygenase-2 inhibitor” or “COX-2 inhibitor” or “cyclooxygenase-II inhibitor” includes agents that specifically inhibit a class of enzymes, COX-2, with less significant inhibition of COX-1. Preferably, it includes compounds that have a COX-2 IC₅₀ of less than about 0.2 μM, and also have a selectivity ratio of COX-2 inhibition over COX-1 inhibition of at least 50, and more preferably of at least 100. Even more preferably, the compounds have a COX-1 IC₅₀ of greater than about 1 μM, and more preferably of greater than 10 μM.

Exemplary COX-2 inhibitors may be selected from the group consisting of celecoxib (SC-58635), DUP-697, flosulide (CGP-28238), meloxicam, 6-methoxy-2 naphthylacetic acid (6-MNA), Vioxx (MK-966), nabumetone (prodrug for 6-MNA), nimesulide, NS-398, SC5766, SC-58215, T-614, deracoxib, valdecoxib, rofecoxib, etoricoxib, 2-(3,5-difluorophenyl)-3[-4-(methylsulfonyl)phenyl]-2-cyclopenten-1-one and 2-(3,4-difluorophenyl)-4-(3-hydroxy-3-methylbutoxy)-5-[4-(methylsulfonyl)phenyl]-3(2H)-pyridazinone; or combinations thereof.

In still other embodiments, the subject intraocular steroids can be conjointly administered with a cyclosporin or a derivative thereof.

(v) Exemplary Formulations

The ocular disorders that can be treated or prevented by the present invention include, but are not limited to: cancerous primary tumors in the eye (e.g., retinoblastoma); ocular neovascularization; ocular edema; ocular inflammation; chronic pain in the eye; endogenous uveitis; Behcet's Disease; corneal transplantation; vernal keratoconjunctivitis; ligneous keratoconjunctivitis; dry eye syndrome; anterior uveitis; onchocerciasis; diseases of the retina; diseases of the retinal pigment epithelium and choroid; retinal degeneration; diabetic retinopathy; closed angle (acute) glaucoma; open angle (chronic) glaucoma; congenital glaucoma; secondary glaucoma; retinal detachment; sickle cell retinopathy; senile macular degeneration; retinal neovascularization; subretinal neovascularization; rubeosis iritis; inflammatory diseases; chronic posterior and panuveitis; neoplasms; pseudoglioma; neovascular glaucoma; neovascularization resulting or following a combined vitrectomy and lensectomy; vascular diseases; retinal ischemia; choroidal vascular insufficiency; choroidal thrombosis; neovascularization of the optic nerve; diabetic macular edema; cystoid macular edema; macular edema; retinitis pigmentosa; retinal vein occlusion; proliferative vitreoretinopathy; angioid streak; retinal artery occlusion; and neovascularization due to penetration of the eye or ocular injury.

In certain embodiments, the subject compositions are derived corticosteroids depleted of endotoxins. Such corticosteroid compounds include dexamethasone, prednisolone, fluocinolone, lotoprednol, triamcinolone, cortisone, flumetholone, and analogs, derivatives, pharmaceutically acceptable salts, esters, prodrugs, codrugs, or protected forms thereof, In certain preferred embodiments, the subject steroid is fluocinolone acetonide, triamcinolone acetonide, or loteprednol etabonate.

In certain embodiments, the subject composition comprises a steroid, such as a corticosteriod, formulated as a suspension that slowly dissolves in ocular fluid, e.g., over a period of more than a day, preferably over a period of at least two or four days, or even over a period of at least a week.

In certain embodiments, the subject intraocular steroids are formulated for sustained release, e.g., for delivery of an effective dose of steroid over a period of time of at least 30 days, and even more preferably at least 3 months, 6 months, or even 12 months. Such sustained release formulations include suspending or otherwise disposing the steroid in a polymer or hydrogel such that the steroid is released slowly as a result of diffusion from the matrix and/or biodegradation of the matrix. Certain exemplary formulations are described in U.S. Patent Application Serial Nos. 60/482677 and 60/501947, the contents of which are hereby incorporated by reference in their entirety. In other embodiments, the steroid is disposed in a sustained release device, e.g., a container, dimensioned for implantation in the eye. Suitable devices of this sort are described in U.S. Pat. Nos. 5,378,475, 5,773,019, 6,001,386, 6,217,895, 6,548,078, 6,375,972, 5,902,598, and 6,331,313, and U.S. patent application Ser. No. 10/714,549, the entire contents of which are incorporated by reference herein.

In one embodiment of the present invention, a sustained release composition or device can be implanted or injected into the eye such that it delivers steroids (such as corticosteroid) to the posterior segment of the eye. In a preferred embodiment, the sustained release composition or device is implanted or injected intravitreally. However, the composition or device may also be implanted or injected in the choroidal space, sub-retinally, or in the sclera. These methods of administration and techniques for their preparation are well known by those of ordinary skill in the art. Methods of administration and techniques for their preparation are set forth in Remington's Pharmaceutical Sciences.

In certain preferred embodiments, the aqueous corticosteroid concentration remains less than the vitreous corticosteroid concentration for substantially the lifetime of the sustained release composition or device. Thus, during release of the corticosteroid, the aqueous corticosteroid concentration is no more than about 0.002 μg/mL to about 0.01 μg/mL.

In certain preferred embodiments, the sustained release composition or device can be prepared to release the corticosteroid by pseudo zero order kinetics with a mean release rate of about 1 μg/day to about 50 μg/day, such as, about 1 μg/day to about 10 μg/day.

In one embodiment of the invention, an ocular device containing fluocinolone acetonide, triamcinolone acetonide, or lotoprednol etabonate as the effective agent in a therapeutically effective amount to reduce, treat, or prevent ocular neovascularization (such as corneal, retinal, or subretinal neovasularization, neovascularization of the optic nerve, neovascularization resulting from a combined vitrectomy and lensectomy, or neovascularization due to penetration of the eye or ocular injury), retinal vein occlusion, diabetic retinopathy, diabetic macular edema, retinitis pigmentosa, chronic posterior or panuveitis, macular or cystoid macular edema, closed angle (acute) glaucoma, open angle (chronic) glaucoma, congenital glaucoma, secondary glaucoma, retinal detachment, sickle cell retinopathy, senile macular degeneration, rubeosis iritis, inflammatory diseases, neoplasms, retinoblastoma, pseudoglioma, neovascular glaucoma, retinal ischemia, choroidal vascular insufficiency, choroidal thrombosis, proliferative vitreoretinopathy, angioid streak, or retinal artery occlusion may be prepared. Endotoxins from such a preparation will be selectively removed using the method of the instant invention as described above so that the overall level of endotoxins is such that the preparation is suitable for intraocular use. Such devices may be used to effectively combat and inhibit undesirable ocular neovascularization, edema, or inflammation when surgically implanted or injected into the vitreous of the eye. Such devices may remain in the vitreous permanently after treatment is complete. The preferred amount of fluocinolone acetonide, triamcinolone acetonide, or loteprednol etabonate used in these devices ranges from about 0.01 mg to about 40 mg. More preferably, such devices contain from about 0.1 mg to about 6 mg of fluocinolone acetonide, triamcinolone acetonide, or lotoprednol etabonate. These preferred ranges of effective components and endotoxins may provide sustained release of the fluocinolone acetonide, triamcinolone acetonide, or lotoprednol etabonate for a period of from several hours to over five years, while minimizing the adverse effects of endotoxins.

While the invention has been described in detail with reference to preferred embodiments thereof, it will be apparent to one skilled in the art that various changes can be made, and equivalents employed, without departing from the scope of the invention. Thus, the invention set forth in the following claims is to be interpreted in the broadest sense allowable by law.

All patents, applications, and references cited above are hereby incorporated by reference in their entirety.

1. A packaged pharmaceutical, comprising a pharmaceutical composition formulated for intraocular injection or implantation as a sustained release device, which composition comprises a therapeutically effective amount of a steroid for use in treating or preventing an ocular disorder, which pharmaceutical composition has an endotoxin concentration of less than 0.3 EU/mL.

2. The packaged pharmaceutical of claim 1, further comprising a label and/or instructions for use of the pharmaceutical composition or device in the treatment or prevention of said ocular disorder.

3. Use of a low endotoxin steroid composition in the manufacture of a medicament for the treatment or prevention of an ocular disorder, which steroid composition has an endotoxin concentration of less than 0.3 EU/mL, and is formulated for intraocular injection or implantation as a sustained release device.

4. A method for treating or preventing an ocular disorder comprising administering to a patient's eye by intraocular injection or implantation of a sustained release device, a steroid composition having an endotoxin concentration of less than 0.3 EU/mL.

5. The packaged pharmaceutical, use, or method of any one of claims 1-4, wherein said ocular disorder is selected from cancerous primary tumors in the eye, (e.g., retinoblastoma); ocular neovascularization; ocular edema; ocular inflammation; chronic pain in the eye; endogenous uveitis; Behcet's Disease; corneal transplantation; vernal keratoconjunctivitis; ligneous keratoconjunctivitis; dry eye syndrome; anterior uveitis; onchocerciasis; diseases of the retina; diseases of the retinal pigment epithelium and choroid; retinal degeneration; diabetic retinopathy; closed angle (acute) glaucoma; open angle (chronic) glaucoma; congenital glaucoma; secondary glaucoma; retinal detachment; sickle cell retinopathy; senile macular degeneration; retinal neovascularization; subretinal neovascularization; rubeosis iritis; inflammatory diseases; chronic posterior or panuveitis; neoplasms; pseudoglioma; neovascular glaucoma; neovascularization resulting or following a combined vitrectomy and lensectomy; vascular diseases retinal ischemia; choroidal vascular insufficiency; choroidal thrombosis; neovascularization of the optic nerve; diabetic macular edema; cystoid macular edema; macular edema; retinitis pigmentosa; retinal vein occlusion; proliferative vitreoretinopathy; angioid streak; retinal artery occlusion; and neovascularization due to penetration of the eye or ocular injury.

6. The packaged pharmaceutical, use, or method of any one of claims 1-4, wherein said steroid is a corticosteroid.

7. The packaged pharmaceutical, use, or method of any one of claims 1-4, wherein said steroid is a corticosteroid selected from: dexamethasone, prednisolone, fluocinolone or fluocinolone acetonide, triamcinolone or triamcinolone acetonide, cortisone, flumetholone, lotepredol etabonate, or analogs, derivatives, pharmaceutically acceptable salts, esters, prodrugs, codrugs, or protected forms thereof.

8. The packaged pharmaceutical, use, or method of any one of claims 1-4, wherein said steroid is triamcinolone acetonide.

9. The packaged pharmaceutical, use, or method of any one of claims 1-4, wherein said steroid is fluocinolone acetonide.

10. The packaged pharmaceutical, use, or method of any one of claims 1-4, wherein said steroid is loteprednol etabonate.

11. The packaged pharmaceutical, use, or method of any one of claims 1-4, wherein said endotoxin concentration is no more than 0.03 EU/mL.

12. The packaged pharmaceutical, use, or method of any one of claims 1-4, wherein said composition is for intraocular injection.

13. The packaged pharmaceutical, use, or method of claim 12, wherein said composition is formulated to deliver an effective dose of steroid in 500 μL or less.

14. The packaged pharmaceutical, use, or method of any one of claims 1-4, wherein said composition has an endotoxin concentration less than 0.1 EU/mg steroid.

15. The packaged pharmaceutical, use, or method of any one of claims 1-4, wherein said composition is co-formulated or conjointly administered with one or more endotoxin inhibitors.

16. The packaged pharmaceutical, use, or method of any one of claims 1-4, wherein said device is co-implanted or co-injected with one or more endotoxin inhibitors.

17. The packaged pharmaceutical, use, or method of claim 15 or 16, wherein said endotoxin inhibitor is a cyclooxygenase (COX) inhibitor, especially a COX-2 inhibitor.

18. The packaged pharmaceutical, use, or method of claim 15 or 16, wherein said endotoxin inhibitor is a cyclosporin or a derivative thereof.

19. The packaged pharmaceutical, use, or method of any one of claims 1-4, wherein said composition or said device is formulated for sustained release, e.g., for delivery of an effective dose of steroid over a period of time of at least 30 days, and even more preferably at least 3 months, 6 months, or even 12 months.

20. The packaged pharmaceutical, use, or method of any one of claims 1-4, wherein said composition or said device is injected or implanted to the posterior segment, the choroidal space, the sub-retina, or the sclera of the eye.

21. The packaged pharmaceutical, use, or method of any one of claims 1-4, wherein said sustained release is a within a period of from several hours to over five years.

22. The method of claim 5, wherein the ocular disorder is selected from ocular neovascularization, retinal vein occlusion, diabetic retinopathy, diabetic macular edema, retinitis pigmentosa, chronic posterior or panuveitis, macular or cystoid macular edema, closed angle (acute) glaucoma, open angle (chronic) glaucoma, congenital glaucoma, secondary glaucoma, retinal detachment, sickle cell retinopathy, senile macular degeneration, rubeosis iritis inflammatory diseases, neoplasms, retinoblastoma, pseudoglioma, neovascular glaucoma, retinal ischemia, choroidal vascular insufficiency, choroidal thrombosis, proliferative vitreoretinopathy, angioid streak, and retinal artery occlusion.

23. The method of claim 22, wherein said steroid is triamcinolone acetonide.

24. The method of claim 22, wherein said steroid is fluocinolone acetonide.

25. The method of claim 22, wherein said steroid is loteprednol etabonate.
